(19) United States
(12) Patent Application Publication
(10) Pub. No.: US 2008/0038302 A1
(43) Pub. Date: Feb. 14, 2008

(54) Collapsible water-containing capsules

(75) Inventor: Kojo Tanaka, Ashiya (JP)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/890,444

(22) Filed: Aug. 6, 2007

Related U.S. Application Data
(60) Provisional application No. 60/836,430, filed on Aug. 8, 2006.

(51) Int. Cl. A61K 8/11; A61K 33/00; A61K 9/48; A61Q 1/00; A61Q 19/00

(52) U.S. Cl. 424/401; 424/456; 424/600

(57) ABSTRACT

Collapsible water-containing capsules comprising by weight: (A) from about 70% to about 92% of a water phase comprising, by weight of the capsule, (1) from about 5% to about 91.9% water and (2) from about 0.1% to about 20% of a gelling agent; (B) from about 1% to about 29.9% of a first pigment component which has a particle size of less than 1 μm and is surface coated with a lipophobic hydrophobic coating material; and (C) from about 0.1% to about 29% of a second pigment component which has a particle size of 1 μm or more, is surface coated with a hydrophobic coating material; and is spherical in shape; wherein the total of the first pigment component and the second pigment component is at least about 8% of the capsule; and wherein the capsule comprises less than 1% of a porous pigments having a particle size of less than 1 μm.

### CROSS REFERENCE TO RELATED APPLICATIONS

This application claims the benefit of U.S. Provisional Application No. 60/836,430 filed on Aug. 8, 2006.

### FIELD OF THE INVENTION

The present invention relates to a collapsible water-containing capsule which is stable under normal storage conditions as well as normal mixing processes, however, collapses upon application on the personal surface. The present invention further relates to methods of making such collapsible capsules, personal care compositions comprising such collapsible capsules, and method of treating or make-up of the skin using such collapsible capsules.

### BACKGROUND

A foundation composition can be applied to the face and other parts of the body to even skin tone and texture and to hide pores, imperfections, fine lines and the like. A foundation composition is also applied to moisturize the skin, to balance the oil level of the skin, and to provide protection against the adverse effects of sunlight, wind, and other environmental factors.

Foundation compositions are generally available in the form of liquid or cream suspensions, emulsions, gels, pressed powders, loose powders or anhydrous oil and wax compositions. Emulsion-type foundations are suitable in that they provide moisturizing effects by the water and water-soluble skin treatment agents incorporated. On the other hand, a larger amount and variation of powders and pigments can be formulated into pressed powders and loose powders.

Recently, consumers who seek moisturization as well as the ideal look having both good coverage and natural look on the skin, have the habit of a two step regimen of foundation application. The two step regimen typically contains application of a liquid or emulsion form foundation followed by a pressed or loose powder foundation. It is conceived by such demanding consumers that such two-step regimen provides best results, however, such regimen is also quite elaborate. There is a need for a foundation product which can provide both good feel and good appearance on the skin.

Meanwhile, collapsible water-containing capsules are known in the art. Such capsules provide a unique feel or change of feel upon application and collapsing on the skin. Upon application to the skin, such capsules provide a moisturizing or fresh feeling. Such capsules may also deliver water-soluble skin active agents such as vitamin C derivatives to the skin, in a more or less stable manner.

Known collapsible water-containing capsules are typically made of fine porous pigments such as silica particles which may or may not be surface treated as disclosed in, for example, PCT Publication WO 01/85138, Japanese Patent Publications 2001-131528A, 2000-247823A, 2000-309506A, 11-130614A, 10-265367A, 5-65212A, and 4-308520A. While the use of silica may provide a relatively stable capsule, it has also been observed that they give a negative dry feeling after application on the skin. This is obviously not preferred for a product that is expected to provide a moisturizing feel due to abundant water contained in the capsule. Some attempts have been made to utilize pigments coated by hydrophobic lipophobic surface coating agents such as disclosed in Japanese Patent Publication 2006-509732A, 2001-226230A, 2001-158716A, and 1-125314A. None of the above mentioned capsules, however, provide a favorable application to the skin while also providing satisfactory shear stress tolerance for incorporating in various product forms. Various product forms include, but are not limited to, powder and liquid foundations.

Based on the foregoing, there is a need for a collapsible water-containing capsule which is capable of providing good feel to the personal surface, while having appropriate shear tolerance such that it is stable under normal storage conditions as well as normal mixing processes, however, collapses upon a certain shear stress upon application on the personal surface. There is further a need for a collapsible water-containing capsule which provides good appearance on the personal surface.

None of the existing art provides all of the advantages and benefits of the present invention.

### SUMMARY

The present invention is directed to a collapsible water-containing capsule comprising by weight:
(a) from about 70% to about 92% of a water phase comprising, by weight of the capsule:
   (1) from about 5% to about 91.9% water, and
   (2) from about 0.1% to about 20% of a gelling agent;
(b) from about 1% to about 29.9% of a first pigment component which has a particle size of less than 1 μm and is surface coated with a lipophobic hydrophobic coating material; and
(c) from about 0.1% to about 29% of a second pigment component which has a particle size of 1 μm or more, is surface coated with a hydrophobic coating material; and is spherical in shape;
   wherein the total of the first pigment component and the second pigment component is at least about 8% of the capsule; and
   wherein the capsule comprises less than 1% of a porous pigments having a particle size of less than 1 μm.

The present invention is also directed to personal care compositions comprising the aforementioned collapsible water-containing capsule.

The present invention is also directed to a method of treating or making up the skin utilizing the aforementioned collapsible water-containing capsule.

The present invention is also directed to a process for making the aforementioned collapsible water-containing capsule.

These and other features, aspects, and advantages of the present invention will become evident to those skilled in the art from a reading of the present disclosure with the appended claims.

### DETAILED DESCRIPTION

While the specification concludes with claims particularly pointing out and distinctly claiming the invention, it is believed that the present invention will be better understood from the following description.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

All ingredients such as actives and other ingredients useful herein may be categorized or described by their cosmetic and/or therapeutic benefit or their postulated mode of action. However, it is to be understood that the active and other ingredients useful herein can, in some instances, provide more than one cosmetic and/or therapeutic benefit or operate via more than one mode of action. Therefore, classifications herein are made for the sake of convenience and are not intended to limit an ingredient to the particularly stated application or applications listed.

### Collapsible Water-Containing Capsule

The present invention is related to a collapsible water-containing capsule which comprises, by weight of the capsule, from about 70% to about 92% of a water phase, among which at least 50% to the capsule is the total of water and optional water-soluble solvents. To hold such abundant amount of aqueous solution in the structure, the capsule of the present invention comprises a gelling agent, a first pigment component, and a second component. The present invention provides a collapsible water-containing capsule which is stable under normal storage conditions as well as normal mixing processes, however, collapses upon application. Such stability for the capsule is believed to be provided by the selection of components as detailed hereafter. Without being bound by theory, it is believed that the gelling agent holds the water phase in a relatively rigid structure, while the first and second pigment components cover the water phase and thereby provide the stability and integrity of the capsule.

Preferably, the capsule of the present invention is substantially free of surfactant. Without being bound by theory, it is believed that surfactants negatively affect the stability and shear stress tolerance of the present capsule by decreasing the surface tension difference between the water phase and the first and second pigment components. Herein, surfactants include those which have detersive capability, as well as those which only act as emulsifiers for emulsifying water and oil phases.

Preferably, the capsule of the present invention comprises less than 1% of porous pigments having a particle size of less than 1 μm. Without being bound by theory, it is believed that porous pigments of small size absorb sebum from the personal surface to such an extent that a dry negative feeling is provided to the personal surface. Porous pigments preferably not comprised at 1% or more in the present invention include silica, aluminum oxide, calcium carbonate, cellulose, and others that may have a porous structure when observed under magnification. It is noted that pigments made from the same chemical compound may take either a porous or non-porous structure, based on the process it is purified, processed, synthesized, or otherwise treated.

The collapsible water-containing capsule of the present invention provides unique benefits on the personal surface, such as skin, hair, or scalp, when collapsed on the surface. It provides an initially fresh, and then moisturizing feel to the surface, by releasing the abundant aqueous solution. The capsule further provides a good feel to the surface by the characteristic of the first and/or second pigment components. When the first and second pigment components are applied on the surface, the components provide the appearance benefits inherent of such pigment components.

The capsule of the present invention may, by itself, provide a product in the form of a loose powder product. The capsule of the present invention may also be mixed with other components to provide different product forms. The capsule of the present invention has appropriate shear tolerance such that it is stable under normal storage conditions, as well as normal mixing process, for example when mixing with the other components, however, collapses upon application to the personal surface.

The collapsible water-containing capsule of the present invention is particularly useful for personal care compositions for delivering water, the pigments, and other components to personal surface. Personal care compositions herein include those for the purpose of skin care, make-up, extensive treatment, perfume, antiperspiration, deodorizing, hair coloring, hair treatment, hair styling, and others. Personal care compositions herein can take the product form of powders, wax solidified solid forms, liquids, lotions, pastes, aerosols, and others. One highly preferred product form embodiment is powder for use on the skin, such as foundation and skin care products.

The capsule of the present invention is particularly suitable for incorporating in personal care compositions for use on the skin, for treatment of the skin, and make-up of the skin. Accordingly, the present invention is also related to a method of treating or making up of the skin comprising the steps of:
(1) providing the collapsible water-containing capsule of the present invention;
(2) shearing the collapsible water-containing capsule on the skin by a finger or an applicator to allow the collapsible water-containing capsule to collapse; whereby the components of the collapsible water-containing capsule are applied on the skin, preferably whereby the components are applied on the skin in the order of vicinity from the skin, water, the first pigment component, and the second pigment component; and
(3) allowing the water to evaporate and/or be absorbed in the skin.

For such personal skin care compositions, the first and second pigment components are selected to provide the appropriate skin treatment and/or make-up benefits. Further, the capsule of the present invention may comprise various skin benefit agents and perfumes in a dissolved or dispersed form in the water phase or attracted within the first and second pigment components. It is advantageous to deliver such skin benefit agents, and perfumes encompassed in the present collapsible water-containing capsule, for one or more reasons. For those components that are heat sensitive, the present capsule prevents or delays evaporation prior to use. For those components that may be deteriorated or compromised in benefit by coming to contact with the remainder of the personal care composition, the present capsules act as a barrier. Other components may provide a certain sensation upon application and collapsing of the present capsule.

### Water Phase

The capsule of the present invention comprises a water phase, the water phase comprising water, gelling agent, and optional water-soluble solvent detailed hereafter. The present capsule comprises, by weight of the capsule, from about 70% to about 92% of the water phase. Water is contained at from about 5% to about 91.9% of the capsule. The water phase may be made only by water and gelling agent. Deionized water is preferably used. Water from natural sources including mineral cations can also be used, depending on the desired characteristic of the product. In one preferred embodiment, water may be sourced from fermented biological cultures or its filtrates. A highly preferred commercial source of this kind is Saccharomycopsis ferment filtrate by the tradename SK-II Pitera available from Kashiwayama.

The pH of the water phase is selected in view of the desired characteristic of the product, and particularly, when skin benefit agents are included, the activity and stability of the skin benefit agents. In one preferred embodiment the pH is adjusted from about 4 to about 8. Buffers and other pH adjusting agents can be included to achieve the desirable pH.

### Water-Soluble Solvent

The water phase of the capsule of the present invention may further comprise a water-soluble solvent selected from lower alkyl alcohols and water-soluble humectants. The water-soluble solvents are selected according to the desired skin feel to be delivered, and/or for delivering certain skin benefit agents.

Lower alkyl alcohols useful herein are monohydric alcohols having 1 to 6 carbons, more preferably ethanol and isopropanol.

Water soluble humectants useful herein include polyhydric alcohols such as butylene glycol (1,3 butanediol), pentylene glycol (1,2-pentanediol), glycerin, sorbitol, propylene glycol, hexylene glycol, ethoxylated glucose, 1,2-hexane diol, 1,2-pentane diol, hexanetriol, dipropylene glycol, erythritol, trehalose, diglycerin, xylitol, maltitol, maltose, glucose, fructose; and other water-soluble compounds such as urea, sodium chondroitin sulfate, sodium hyaluronate, sodium adenosin phosphate, sodium lactate, pyrrolidone carbonate, glucosamine, cyclodextrin, and mixtures thereof. Also useful herein include water soluble alkoxylated nonionic polymers such as polyethylene glycols and polypropylene glycols having a molecular weight of up to about 1000 such as those with CTFA names PEG-200, PEG-400, PEG-600, PEG-1000, and mixtures thereof.

In one preferred embodiment, the present capsule comprises from about 1% to about 30% of a water-soluble humectant. In one highly preferred embodiment wherein the capsule is used as a foundation, the capsule comprises from about 3% to about 30% of a water-soluble humectant.

Commercially available humectants herein include: butylene glycol with tradename 1,3-Butylene glycol available from Celanese, pentylene glycol with tradename HYDROLITE-5 available from Dragoco, glycerin with tradenames STAR and SUPEROL available from The Procter & Gamble Company, CRODEROL GA7000 available from Croda Universal Ltd., PRECERIN series available from Unichema, and a same tradename as the chemical name available from NOF; propylene glycol with tradename LEXOL PG-865/855 available from Inolex, 1,2-PROPYLENE GLYCOL USP available from BASF; sorbitol with tradenames LIPONIC series available from Lipo, SORBO, ALEX, A-625, and A-641 available from ICI, and UNISWEET 70, UNISWEET CONC available from UPI; dipropylene glycol with the same tradename available from BASF; diglycerin with tradename DIGLYCEROL available from Solvay GmbH; xylitol with the same tradename available from Kyowa and Eizai; maltitol with tradename MALBIT available from Hayashibara, sodium chondroitin sulfate with the same tradename available from Freeman and Bioiberica, and with tradename ATOMERGIC SODIUM CHONDROITIN SULFATE available from Atomergic Chemetals; sodium hyaluronate available from Chisso Corp, the same with tradenames ACTIMOIST available from Active Organics, AVIAN SODIUM HYALURONATE series available from Intergen, HYALURONIC ACID Na available from Ichimaru Pharcos; sodium adenosin phophate with the same tradename available from Asahikasei, Kyowa, and Daiichi Seiyaku; sodium lactate with the same tradename available from Merck, Wako, and Showa Kako, cyclodextrin with tradenames CAVITRON available from American Maize, RHODOCAP series available from Rhone-Poulenc, and DEXPEARL available from Tomen; and polyethylene glycols with the tradename CARBOWAX series available from Union Carbide.

### Gelling Agents

The collapsible water-containing capsule of the present composition comprises, by weight of the capsule, from about 0.1% to about 20%, preferably from about 0.1% to about 5%, of a gelling agent that provides the water phase a viscosity of from about 10 mPas to about 1,000,000 mPas, preferably from about 10 mPas to about 100,000 mPas. The gelling agent holds water and optional water-soluble solvents in a relatively rigid structure, and thereby believed to provide the stability and integrity of the capsule.

The polymers useful as the gelling agent herein are water soluble or water miscible polymers. The term “water soluble or water miscible” with regard to the gelling agents herein, relate to compounds that are dissolved to make a transparent solution when dissolved in ample amount of water with or without the aid of elevated temperature and/or mixing.

Useful herein are starch derivative polymers such as carboxymethyl starch, and methylhydroxypropyl starch. Commercially available compounds that are highly useful herein include sodium carboxymethyl starch with tradename COVAGEL available from LCW.

Useful herein are cellulose derivative polymers. Cellulose derivative polymers useful herein include methylcellulose, ethylcellulose, hydroxyethylcellulose, hydroxyethyl ethylcellulose, hydroxypropyl methyl cellulose, nitrocellulose, sodium cellulose sulfate, sodium carboxymethylcellulose, crystalline cellulose, cellulose powder, and mixtures thereof. Also useful are starch derivative polymers such as carboxymethyl starch, and methylhydroxypropyl starch. Commercially available compounds that are highly useful herein include hydroxyethylcellulose with tradename Natrosol Hydroxyethylcellulose, and carboxymethylcellulose with tradename Aqualon Cellulose Gum, both available from Aqualon.

Useful herein are carboxylic acid/carboxylate copolymers. Commercially available carboxylic acid/carboxylate copolymers useful herein include: CTFA name Acrylates/C10-30 Alkyl Acrylate Crosspolymer having tradenames Pemulen TR-1, Pemulen TR-2, Carbopol 1342, Carbopol 1382, and Carbopol ETD 2020, all available from B. F. Goodrich Company.

Neutralizing agents may be included to neutralize the carboxylic acid/carboxylate copolymers herein. Nonlimiting examples of such neutralizing agents include sodium hydroxide, potassium hydroxide, ammonium hydroxide, monoethanolamine, diethanolamine, triethanolamine, diisopropanolamine, aminomethylpropanol, tromethamine, tetrahydroxypropyl ethylenediamine, and mixtures thereof.

Polyalkylene glycols having a molecular weight of more than about 1000 are useful herein. Useful are those having the following general formula:

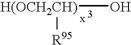

wherein R⁹⁵ is selected from the group consisting of H, methyl, and mixtures thereof. When R⁹⁵ is H, these materials are polymers of ethylene oxide, which are also known as polyethylene oxides, polyoxyethylenes, and polyethylene glycols. When R⁹⁵ is methyl, these materials are polymers of propylene oxide, which are also known as polypropylene oxides, polyoxypropylenes, and polypropylene glycols. When R⁹⁵ is methyl, it is also understood that various positional isomers of the resulting polymers can exist. In the above structure, ×3 has an average value of from about 1500 to about 25,000, preferably from about 2500 to about 20,000, and more preferably from about 3500 to about 15,000. Other useful polymers include the polypropylene glycols and mixed polyethylene-polypropylene glycols, or polyoxyethylene-polyoxypropylene copolymer polymers. Polyethylene glycol polymers useful herein are PEG-2M wherein R⁹⁵ equals H and ×3 has an average value of about 2,000 (PEG-2M is also known as Polyox WSR® N-10, which is available from Union Carbide and as PEG-2,000); PEG-5M wherein R⁹⁵ equals H and ×3 has an average value of about 5,000 (PEG-5M is also known as Polyox WSR® N-35 and Polyox WSR® N-80, both available from Union Carbide and as PEG-5,000 and Polyethylene Glycol 300,000); PEG-7M wherein R⁹⁵ equals H and ×3 has an average value of about 7,000 (PEG-7M is also known as Polyox WSR® N-750 available from Union Carbide); PEG-9M wherein R⁹⁵ equals H and ×3 has an average value of about 9,000 (PEG 9-M is also known as Polyox WSR® N-3333 available from Union Carbide); and PEG-14 M wherein R⁹⁵ equals H and ×3 has an average value of about 14,000 (PEG-14M is also known as POLYOX WSR® N-3000 available from Union Carbide).

Useful herein are vinyl polymers such as cross linked acrylic acid polymers with the CTFA name Carbomer, pullulan, mannan, scleroglucans, polyvinylpyrrolidone, polyvinyl alcohol, guar gum, hydroxypropyl guar gum, xanthan gum, acacia gum, arabia gum, tragacanth, galactan, carob gum, karaya gum, locust bean gum, carrageenin, pectin, amylopectin, agar, quince seed (Cyclonia oblonga Mill), starch (rice, corn, potato, wheat), algae colloids (algae extract), microbiological polymers such as dextran, succinoglucan, starch-based polymers such as carboxymethyl starch, methylhydroxypropyl starch, alginic acid-based polymers such as sodium alginate, alginic acid propylene glycol esters, acrylate polymers such as sodium polyacrylate, polyacrylamide, polyethyleneimine, and inorganic water soluble material such as bentonite, aluminum magnesium silicate, laponite, hectonite, and anhydrous silicic acid.

Commercially available gelling agents useful herein include xanthan gum with tradename KELTROL series available from Kelco, Carbomers with tradenames CARBOPOL 934, CARBOPOL 940, CARBOPOL 950, CARBOPOL 980, and CARBOPOL 981, all available from B. F. Goodrich Company, acrylates/steareth-20 methacrylate copolymer with tradename ACRYSOL 22 available from Rohm and Hass, polyacrylamide with tradename SEPIGEL 305 available from Seppic, glyceryl polymethacrylate with tradename LUBRAGEL NP, and a mixture of glyceryl polymethacrylate, propylene glycol and PVM/MA copolymer with tradename LUBRAGEL OIL available from ISP, scleroglucan with tradename Clearogel SC11 available from Michel Mercier Products Inc. (NJ, USA), ethylene oxide and/or propylene oxide based polymers with tradenames CARBOWAX PEGs, POLYOX WASRs, and UCON FLUIDS, all supplied by Amerchol.

Useful herein are amphoteric polymers such as Polyquaternium 22 with tradenames MERQUAT 280, MERQUAT 295, Polyquaternium 39 with tradenames MERQUAT PLUS 3330, MERQUAT PLUS 3331, and Polyquaternium 47 with tradenames MERQUAT 2001, MERQUAT 2001N, all available from Calgon Corporation. Other useful amphoteric polymers include octylacrylamine/acrylates/butylaminoethyl methacrylate copolymers with the tradenames AMPHOMER, AMPHOMER SH701, AMPHOMER 28-4910, AMPHOMER LV71, and AMPHOMER LV47 supplied by National Starch & Chemical.

### First Pigment Component

The collapsible water-containing capsule of the present composition comprises, by weight of the capsule, from about 1% to about 29.9%, preferably from about 6% to about 27%, still preferably from about 8% to about 25% of a first pigment component. The first pigment component herein has a particle size of less than 1 μm, preferably from about 5 nm to about 600 nm, more preferably from about 10 nm to about 500 nm, and is surface coated with a lipophobic hydrophobic coating material. Without being bound by theory, it is believed that, by the surface tension of the lipophobic hydrophobic surface of the first pigment component, the first pigment component aligns at the phase boundary of the water phase, while the particles of the first pigment component bind with each other via van-der-Waals binding. Hence, the first pigment component covers the water phase. It is further believed that the overall structure due to the lipophobic hydrophobic surface, combined with the relatively small particle size of the first pigment component, contributes to the suitable shear stress tolerance of the collapsible water-containing capsule of the present composition. In an highly preferred embodiment where the present capsule comprises from about 6% to about 27% of the first pigment component, the stability of the capsule is enhanced.

The base pigments of the first pigment component useful herein include those that provide color or change tone, and also those that provide a certain skin feel. Useful pigments herein include clay mineral powders such as talc, magnesium silicate, synthetic fluorphlogopite, calcium silicate, aluminum silicate, bentonite and montomorilonite. The coloring powders useful herein include pearl pigments such as alumina, barium sulfate, calcium secondary phosphate, zirconium oxide, zinc oxide, hydroxy apatite, iron oxide, iron titate, ultramarine blue, Prussian blue, chromium oxide, chromium hydroxide, cobalt oxide, cobalt titanate, titanium oxide coated mica; organic powders such as polyester, polyethylene, polystyrene, methyl metharylate resin, 12-nylon, 6-nylon, styrene-acrylic acid copolymers, poly propylene, vinyl chloride polymer, tetrafluoroethylene polymer, boron nitride, fish scale guanine, laked tar color dyes, and laked natural color dyes. Particularly useful herein as the first pigment component are titanium dioxide, zinc oxide, iron oxide, barium sulfate, polystyrene, and mixtures thereof.

The first pigment component herein is surface coated with a coating material having both lipophobic and hydrophobic characteristics, such as fluorine compounds. Particularly suitable fluorine compounds herein are selected from the group consisting of perfluorooctyl triethoxylsilane, perfluoroalkylphosphoric acids, their salts, and mixtures thereof.

Commercially available first pigment components highly useful herein include Titanium Dioxide coated with C9-15 fluoroalcohol phosphates (0.25 μm) with tradename PF-5 TiO2 CR-50, Titanium Dioxide coated with perfluorooctyl triethoxysilane (0.021 μm) with tradename FHS-12 TiO2 P-25, Zinc Oxide coated with C9-15 fluoroalcohol phosphates (0.020 μm) with tradename PF-7 ZnO-350, Yellow Iron Oxide coated with C9-15 fluoroalcohol phosphates (0.435 μm) with tradename PF-5 YELLOW LL-100PD, Red Iron Oxide coated with C9-15 fluoroalcohol phosphates (0.44 μm) with tradename PF-5 RED R-516PD, and Black Iron Oxide coated with C9-15 fluoroalcohol phosphates (0.4 μm) with tradename PF-5 BLACK BL-100P, all available from Daito Kasei.

### Second Pigment Component

The collapsible water-containing capsule of the present composition comprises, by weight of the capsule, from about 0.1% to about 29%, preferably from about 1% to about 10%, of a second pigment component. The second pigment component herein has a particle size of 1 μm or more, preferably from about 1 μm to about 25 μm, more preferably from about 4 μm to about 15 μm, is surface coated with a hydrophobic coating material, and is spherical in shape. Without being bound by theory, it is believed that, by the larger size and spherical shape of the second pigment component, the second pigment component aligns at the phase boundary of the first pigment component. It is believed that the dual covered structure provided by the first and second pigment components provide the suitable shear stress tolerance of the collapsible water-containing capsule of the present composition.

The total of the first pigment component and the second pigment component is at least about 8% of the capsule, preferably from about 8% to about 26% of the capsule.

The second pigment component also provides a unique appearance effect or skin feel that is not easily delivered by the first pigment component. In one example, the first pigment components alone may provide an overly matte finish and emphasize, rather than hide, skin unevenness such as pores. A spherical and translucent second pigment component can improve the natural appearance by light diffusion effect due to its shape and translucency. In another example, the first pigment components alone may provide a squeaky feel on the skin due to their small size. A soft spherical second pigment component may alleviate such negative skin feel and provide good smooth feel.

The base pigments of the second pigment component useful herein include; polyacrylates, silicates, sulfates, alumina, metal dioxides, carbonates, celluloses, polyalkylenes, vinyl acetates, polystyrenes, polyamides, acrylic acid ethers, silicones, and mixtures and complexes thereof. Specifically, materials useful herein include polyacrylates such as methyl methacrylate copolymer and nylon, cross linked polymethyl methacrylate; silicates such as calcium silicate, magnesium silicate, barium silicate, aluminium silicate and silica beads; alumina; metal dioxides such as titanium dioxide and aluminium hydroxide; carbonates such as calcium carbonate, magnesium carbonate; celluloses; polyalkylenes such as polyethylene, and polypropylene; vinyl acetates; polystyrenes; polyamides; acrylic acid ethers such as acrylic acid methyl ether and acrylic acid ethyl ether; polyvinyl pyrrolidones; and silicones such as polyorganosilsesquioxane resin such as polymethyl silsequioxane and solid silicone elastomers such as vinyl dimethicone/methicone silsesquioxane crosspolymer. Highly preferred materials are selected from the group consisting of methyl methacrylate copolymer, silica beads, nylon, polymethyl silsesquioxane, vinyl dimethicone/methicone silsesquioxane crosspolymer, polyorganosiloxane elastomer, and mixtures thereof.

In one embodiment, polyorganosilsesquioxane resin and solid silicone elastomers may be used for enhancing the effect of hiding skin pores.

The second pigment component herein is surface coated with a coating material having hydrophobic characteristics, whereby lipophobic hydrophobic coating materials are preferred. Useful hydrophobic coating materials herein include methyl polysiloxane, methyl hydrogen polysiloxane, methyl phenyl polysilxoane, n-octyl triethoxy silane, methyl-alpha-styrene polysiloxane, acryl silicone copolymer, and mixtures thereof. Preferred lipophobic hydrophobic coating materials are the same coating material as aforementioned for the first pigment component.

Commercially available spherical powders highly useful herein include methyl methacylate copolymer with tradename GANZ PEARL series available from Ganz Chemical Co., Ltd., and SYLYSIA series available from Fuji Sylysia Chemical, Nylon-12 with tradename NYLON POWDER series available from Toray Dow Corning, Nylon-12 coated with C9-15 fluoroalcohol phosphates (5 μm) with tradename PF-5 NYLON SP 500 available from Daito Kasei, polymethyl silsesquioxiane coated with C9-15 fluoroalcohol phosphates with tradename PF-5 TOSPEARL 145 available from Daito Kasei, vinyl dimethicone/methicone silsesquioxane crosspolymer with tradenames KSP series available from ShinEtsu Chemical Co., Ltd., Tokyo Japan, and hardened polyorgano siloxane elastomers with tradenames TREFIL series available from Toray Dow Corning.

### Skin Benefit Agent

The capsule of the present composition may further comprise a skin benefit agent dissolved or dispersed in the water phase or the pigment components. Those skin benefit agents of polar nature can be dissolved or dispersed in the water phase, while those that do not dissolve or disperse in the water phase may be mixed and attracted within the pigment components. When included, the skin benefit agent is included in an amount that does not affect the stability of the capsule, typically by weight of the capsule, at from about 0.001% to about 20%.

The skin benefit agents useful herein include skin lightening agents, anti-acne agents, emollients, non-steroidal anti-inflammatory agents, topical anesthetics, artificial tanning agents, antiseptics, anti-microbial and anti-fungal actives, skin soothing agents, UV protection agents, skin barrier repair agents, anti-wrinkle agents, anti-skin atrophy actives, lipids, sebum inhibitors, sebum inhibitors, skin sensates, protease inhibitors, skin tightening agents, anti-itch agents, hair growth inhibitors, desquamation enzyme enhancers, anti-glycation agents, antiperspirant actives, oxidative hair colorants, hair styling agents, and mixtures thereof.

Commercially available flavonoid compounds include hesperidin, methylhesperidin, and rutin available from Alps Pharmaceutical Industry Co. Ltd. (Japan); and glucosyl hesperidin with tradename α-Ghesperidin PS-CC and glucosyl rutin available from Hayashibara Biochemical Laboratories, Inc. (Japan) and Toyo Sugar Refining Co. Ltd. (Japan).

Vitamin B3 compounds useful herein include, for example, those having the formula:

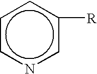

wherein R is —CONH₂ (e.g., niacinamide) or —CH₂OH (e.g., nicotinyl alcohol); derivatives thereof; and salts thereof. Exemplary derivatives of the foregoing vitamin B₃ compounds include nicotinic acid esters, including non-vasodilating esters of nicotinic acid, nicotinyl amino acids, nicotinyl alcohol esters of carboxylic acids, nicotinic acid N-oxide and niacinamide N-oxide. Preferred vitamin B₃ compounds are niacinamide and tocopherol nicotinate, and more preferred is niacinamide. In a preferred embodiment, the vitamin B₃ compound contains a limited amount of the salt form and is more preferably substantially free of salts of a vitamin B₃ compound. Preferably the vitamin B₃ compound contains less than about 50% of such salt, and is more preferably essentially free of the salt form. Commercially available vitamin B₃ compounds that are highly useful herein include niacinamide USP available from Reilly.

Vitamin B6 compounds useful herein include pyridoxine; esters of pyridoxine such as pyridoxine tripahnitate, pyridoxine dipalmitate, and pyridoxine dioctanoate; amines of pyridoxine such as pyridoxamine; salts of pyridoxine such as pyridoxine HCl; and derivatives thereof such as pyridoxamine, pyridoxal, pyridoxal phosphate, and pyridoxic acid. Particularly useful vitamin B6 compounds are selected from the group consisting of pyridoxine, esters of pyridoxine and salts of pyridoxine. The vitamin B6 compound can be synthetic or natural in origin and can be used as an essentially pure compound or mixtures of compounds (e.g., extracts from natural sources or mixtures of synthetic materials). As used herein, “vitamin B6” includes isomers and 6 tautomers of such. Commercially available vitamin B6 compound useful herein include, for example, pyridoxine HCl available from DSM, pyridoxine dipalmitate with tradename NIKKOL DP and pyridoxine dioctanoate with tradename NIKKOL DK available from Nikko Chemicals Co. Ltd.

Skin lightening agents useful herein refer to active ingredients that improve hyperpigmentation as compared to pre-treatment. Useful skin lightening agents herein include ascorbic acid compounds, azelaic acid, butyl hydroxyanisole, gallic acid and its derivatives, glycyrrhizinic acid, hydroquinone, kojic acid, arbutin, mulberry extract, and mixtures thereof. Use of combinations of skin lightening agents is believed to be advantageous in that they may provide skin lightening benefit through different mechanisms.

Ascorbic acid compounds useful herein include, ascorbic acid per se in the L-form, ascorbic acid salt, and derivatives thereof. Ascorbic acid is available from, for example, Roche Vitamins Japan. Ascorbic acid salts useful herein include, sodium, potassium, lithium, calcium, magnesium, barium, ammonium and protamine salts. Ascorbic acid derivatives useful herein include, for example, esters of ascorbic acid, and ester salts of ascorbic acid. Particularly preferred ascorbic acid compounds include 2-o-D-glucopyranosyl-L-ascorbic acid, which is an ester of ascorbic acid and glucose and usually referred to as L-ascorbic acid 2-glucoside or ascorbyl glucoside, and its metal salts, and L-ascorbic acid phosphate ester salts such as sodium ascorbyl phosphate, potassium ascorbyl phosphate, magnesium ascorbyl phosphate, and calcium ascorbyl phosphate. Commercially available ascorbic compounds include magnesium ascorbyl phosphate available from Showa Denko, 2-o-D-glucopyranosyl-L-ascorbic acid available from Hayashibara and sodium L-ascorbyl phosphate with tradename STAY C50 available from DSM.

Other hydrophobic skin lightening agents useful herein include ascorbic acid derivatives such as ascorbyl tetraisopalmitate (for example, VC-IP available from Nikko Chemical), ascorbyl palmitate (for example available from DSM), ascorbyl dipalmitate (for example, NIKKOL CP available from Nikko Chemical); undecylenoyl phenyl alanine (for example, SEPIWHITE MSH available from Seppic); octadecenedioic acid (for example, ARLATONE DIOIC DCA available from Uniquema); oenothera biennis sead extract, and pyrus malus (apple) fruit extract, and mixtures thereof.

Other skin benefit agents useful herein include those selected from the group consisting of panthenol, benzoyl peroxide, 3-hydroxy benzoic acid, farnesol, phytantriol, glycolic acid, lactic acid, 4-hydroxy benzoic acid, acetyl salicylic acid, 2-hydroxybutanoic acid, 2-hydroxypentanoic acid, 2-hydroxyhexanoic acid, cis-retinoic acid, trans-retinoic acid, retinol, retinyl esters (e.g., retinyl propionate), phytic acid, N-acetyl-L-cysteine, lipoic acid, tocopherol and its esters (e.g., tocopheryl acetate), azelaic acid, arachidonic acid, tetracycline, ibuprofen, naproxen, ketoprofen, hydrocortisone, acetominophen, resorcinol, phenoxyethanol, phenoxypropanol, phenoxyisopropanol, 2,4,4′-trichloro-2′-hydroxy diphenyl ether, 3,4,4′-trichlorocarbanilide, octopirox, lidocaine hydrochloride, clotrimazole, miconazole, ketoconazole, neomycin sulfate, theophylline, and mixtures thereof.

UV protection agents for providing sunlight and UV protection benefit are useful as skin benefit agents herein. When included, the total of organic UV protection agent is from about 0.1% to about 20% of the capsule. Oil-soluble organic UV agents, water-soluble organic UV agents, and inorganic UV agents may be incorporated in the capsule of the present invention. Inorganic UV protection agents have a particle size of smaller than 200 nm, preferably smaller than 100 nm. Thus, depending on the surface coating characteristic, certain first pigment components may provide UV protection benefit. Those that are not surface coated with lipophilic hydrophobic coating material are also useful herein as UV protection agents that disperse in the water phase. Useful organic UV protection agents include both those which absorb UV radiation mainly in the UVB range, and those which absorb UV radiation mainly in the UVA range. Protection from UVB is described by SPF (Sun Protection Factor) and UVA is described by PA (Protection of UVA). It is well known in the art that combining UVA and UVB protection agents provide a composition having effective sunscreen effect. In one preferred embodiment, the present invention is a sunscreen product or a cosmetic product having an SPF of at least 15 and a PA of at least ++.

Useful oil-soluble organic UV protection agents effective as UVB filters include: 3-benzylidenecamphor derivatives, preferably 3-(4-methylbenzylidene) camphor and 3-benzylidenecamphor; aminobenzoic acid derivatives, preferably 2-ethylhexyl 4-(dimethylamino)-benzoate and amyl 4-(dimethylamino) benzoate; esters of cinnamic acid, preferably 2-ethylhexyl 4-methoxycinnamate and isopentyl 4-methoxycinnamate; esters of salicylic acid, preferably 2-ethylhexyl salicylate, 4-isopropylbenzyl salicylate and homomethyl salicylate; derivatives of benzophenone, preferably 2-hydroxy-4-methoxybenzophenone (Benzophenone-3), 2-hydroxy-4-methoxy-4′-methylbenzophenone and 2.2′-dihydroxy-4-methoxybenzophenone; esters of benzalmalonic acid, preferably di(2-ethylhexyl) 4-methoxybenzalmalonate; and 2,4,6-trianilino-(p-carbo-2′-ethyl-1′-hexyloxy)-1,3,5-triazine.

Useful oil-soluble organic UV protection agents effective as UVA filters include: derivatives of dibenzoylmethane, in particular 1-(4′-tert-butylphenyl)-3-(4′-methoxyphenyl) propane-1,3-dione and 1-phenyl-3-(4′-isopropylphenyl) propane-1,3-dione.

Commercially available oil-soluble organic UV protection agents herein include: 2-ethylhexyl 4-methoxycinnamate with tradename PARSOL MCX available from ROCHE VITAMINS JAPAN K.K and 2-hydroxy-4-methoxybenzophenone (Benzophenone-3) available from BASF.

Useful water-soluble organic UV protection agents effective as UVB filters include: 2-phenylbenzimidazole-5-sulphonic acid, and its sodium, potassium or its triethanol-ammonium salts; sulphonic acid derivatives of benzophenones, preferably 2-hydroxy-4-methoxybenzophenone-5-sulphonic acid (Benzophenone-4) and its salts; sulphonic acid derivatives of 3-benzylidenecamphor, such as, for example 4-(2-oxo-3-bornylidenemethyl)-benzenesulphonic acid, 2-methyl-5-(2-oxo-3-bornylidenemethyl) sulphonic acid and its salts.

Commercially available water-soluble organic UV protection agents herein include: phenylbenzimidazole-5-sulphonic acid with tradename PARSOL HS available from BASF and Neo Helopan Hydro available from Symrise, and 2-hydroxy-4-methoxybenzophenone-5-sulphonic acid (Benzophenone-4) available from BASF.

Useful inorganic UV protection agents herein are cosmetic and dermatological acceptable metal oxides and/or other metal compounds which are sparingly soluble or insoluble in water, in particular the oxides of titanium (TiO₂), zinc (ZnO), iron (for example Fe₂O₃), zirconium (ZrO₂), silicon (SiO₂), manganese (for example MnO), aluminum (Al₂O₃) and cerium (for example Ce₂O₃), mixed oxides of the corresponding metals and mixtures of such oxides.

The modifications in which such metal oxides are present are essentially irrelevant to the present invention. For example, TiO₂ occurs in nature in three main modifications (rutile, anatase and brookite), which in principle are all equally suitable. The same applies to the modifications of iron oxides and the like.

Inorganic UV protection agents herein may be given a water-repellent treatment on the surface. This surface treatment can comprise providing the pigments with a thin hydrophobic layer by processes known per se. Such a process may be producing the hydrophobic surface layer by a reaction according to nTiO₂+m(RO)₃Si—R′→nTiO₂ (surf.). n and m here are stoichiometric parameters to be inserted as required, and R and R′ are the desired organic radicals. Hydrophobized pigments prepared analogously to DE-A 33 14 742, for example, are of advantage.

Commercially available inorganic UV protection agents herein include: zinc oxide having an average particle size of about 70 nm with tradename Z-cote HP1 available from BASF, and titanium oxide having an average particle size of about 50 nm with tradenames SI-TTO-S-3Z-LHC and SAMT-UFZO-450 available from Miyoshi.

### Additional Components

The capsules hereof may further contain additional components such as are conventionally used in topical products, e.g., for providing aesthetic or functional benefit to the composition or personal surface, such as sensory benefits relating to appearance, smell, or feel, therapeutic benefits, or prophylactic benefits (it is to be understood that the above-described required materials may themselves provide such benefits). When included, the amount is kept to no more than about 10% by weight of the capsule.

Examples of suitable topical ingredient classes include: powders and pigments that do not meet the definition of other pigment or inorganic UV protection agents above, anti-chelating agents, abrasives, astringents, dyes, essential oils, fragrance, film forming polymers, solubilizing agents, anti-caking agents, antifoaming agents, binders, buffering agents, bulking agents, denaturants, pH adjusters, propellants, reducing agents, sequestrants, cosmetic biocides, and preservatives.

### Process for Making the Collapsible Water-Containing Capsule

The capsule of the present invention is suitably made by mixing the essential components of the capsule described hereinabove. It has been surprisingly and unexpectedly discovered that the capsules of the present invention can be made without extreme conditions and steps disclosed in the art for providing water-containing capsules. Extreme conditions and steps include, but are not limited to, high shear mixing, freezing prior to shearing, and others.

In one embodiment, the collapsible water-containing capsule of the present invention is prepared by the steps of:

(1) uniformly mixing the gelling agent, the first pigment component and the second pigment component; and
(2) mixing the product of step (1) with the water phase.

In another embodiment, the collapsible water-containing capsule of the present invention is prepared by the steps of:

(1) uniformly mixing the gelling agent and the first pigment component;
(2) mixing the product of step (1) with the water phase; and
(3) mixing the product of step (2) with the second pigment component.

In one highly preferred embodiment suitable for small scale production, the gelling agent, first and second pigments are mixed in a vessel, and further mixed with water in a container that has a hydrophobic inner surface.

In another highly preferred embodiment suitable for larger scale production, the gelling agent, first and second pigments are mixed in a pulverizer, and further mixed in a ribbon blender, where water is sprayed.

### Shear Tolerance of the Collapsible Water-Containing Capsule

The capsule of the present invention has appropriate shear tolerance such that it is stable under normal storage conditions as well as normal mixing processes, however, collapses upon application on the personal surface. The collapsing of the present capsule can be easily observed by the naked eye, as a flowable dry powdery appearance of the original capsule is changed to a non-flowable wet pasty appearance.

Such shear tolerance is suitably quantitatively measured by the Drop Test as herein defined:

1) 40 g of collapsible water-containing capsule of the present invention is transferred in a 100 ml translucent plastic bottle which is sealable via a cap;
2) the test container thus prepared is sealed via the cap, and dropped from a given height on a concrete surface; and
3) the dropped test container is observed by the naked eye to check if any capsules are collapsed.

The given height of step 2) indicates the quantitative shear tolerance of The Drop Test in units of cm.

The collapsible water-containing capsules of the present invention have a shear tolerance of at least 100 cm according to The Drop Test.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

The following are capsule compositions for use on skin, method of preparation thereof, and technical assessment of their characteristics thereof. Examples 1-5 are capsules according to the present invention, while Comparative Examples 1-7 are those that are not according to the present invention.

TABLE 1
Composition for Examples 1-4

| | | Ex. 1 | Ex. 2 | Ex. 3 | Ex. 4 |
|---|---|---|---|---|---|
| 1 | Titanium Dioxide coated with C9-15 | 10 | 10 | | |
| | fluoroalcohol phosphates (0.25 μm) *1 | | | | |
| 2 | Titanium Dioxide coated with perfluorooctyl | | | 8.15 | 10 |
| | triethoxysilane (0.021 μm) *2 | | | | |
| 3 | Zinc Oxide coated with C9-15 fluoroalcohol | | | 5 | |
| | phosphates (0.020 μm) *3 | | | | |
| 4 | Yellow Iron Oxide coated with C9-15 | 2 | 2 | 0.2 | |
| | fluoroalcohol phosphates (0.435 μm) *4 | | | | |
| 5 | Red Iron Oxide coated with C9-15 fluoroalcohol | 1 | 1 | 0.1 | |
| | phosphates (0.44 μm) *5 | | | | |
| 6 | Black Iron Oxide coated with C9-15 | 0.5 | 0.5 | 0.05 | |
| | fluoroalcohol phosphates (0.4 μm) *6 | | | | |
| 7 | Sodium Carboxymethyl Starch *7 | 1.5 | | 1.5 | 1.5 |
| 8 | Xanthan Gum *8 | | 1 | | |
| 9 | Nylon-12 coated with C9-15 fluoroalcohol | 5 | | 4.5 | 7.7 |
| | phosphates (5 μm) *9 | | | | |
| 10 | Polymethyl Silsesquioxiane coated with C9-15 | | 4.5 | | |
| | fluoroalcohol phosphates (4.5 μm) *10 | | | | |
| 11 | Mica (10 μm) *11 | | 1 | | 1.85 |
| 12 | Titanium Dioxide (0.040 μm) *12 | | | 2.7 | |
| 13 | Ascorbic Acid *13 | | | | 1 |
| 14 | D-delta-tocopherol *14 | | | | 0.1 |
| 15 | Ethylhexyl Methoxycinnamate *15 | | | 0.3 | |
| 16 | Butylene Glycol *16 | 15 | 15 | 10 | |
| 17 | Glycerin | | | | 5 |
| 18 | Ethanol | | | | 2 |
| 19 | Titanium Dioxide (0.035 μm) *17 | | | 3 | |
| 20 | Mica coated with Titanium Dioxide *18 | | 2 | | |
| 21 | Glucosyl Hesperidin *19 | | | | 0.5 |
| 22 | Niacinamide *20 | | | | 2 |
| 23 | Mulberry Root Extract *21 | | | 5 | |
| 24 | Panthenol *22 | | | | 1 |
| 25 | Saccharomycopsis Ferment Filtrate *23 | | | | 10 |
| 26 | WATER | 64.5 | 62.5 | 59 | 56.7 |
| 27 | EDTA-2NA | | | | 0.1 |
| 28 | PRESERVATIVES | 0.5 | 0.5 | 0.5 | 0.5 |
| 29 | Perfume | | | | 0.05 |
| | Total | 100 | 100 | 100 | 100 |

Definitions of Components
*1 Titanium Dioxide coated with C9-15 fluoroalcohol phosphates (0.25 μm): PF-5 TiO2 CR-50 available from Daito Kasei.
*2 Titanium Dioxide coated with perfluorooctyl triethoxysilane (0.021 μm): FHS-12 TiO2 P-25 available from Daito Kasei.
*3 Zinc Oxide coated with C9-15 fluoroalcohol phosphates (0.020 μm): PF-7 ZnO-350 available from Daito Kasei.
*4 Yellow Iron Oxide coated with C9-15 fluoroalcohol phosphates (0.435 μm): PF-5 YELLOW LL-100PD available from Daito Kasei.
*5 Red Iron Oxide coated with C9-15 fluoroalcohol phosphates (0.44 μm): PF-5 RED R-516PD available from Daito Kasei.
*6 Black Iron Oxide coated with C9-15 fluoroalcohol phosphates (0.4 μm): PF-5 BLACK BL-100P available from Daito Kasei.
*7 Sodium Carboxymethyl Starch: COVAGEL available from LCW.
*8 Xanthan Gum: Keltrol T available from Kelco.
*9 Nylon-12 coated with C9-15 fluoroalcohol phosphates (5 μm): PF-5 NYLON
*10 Polymethyl Silsesquioxiane coated with C9-15 fluoroalcohol phosphates (4.5 μm): PF-5 TOSPEARL 145 available from Daito Kasei.
*11 Mica (10 μm): SERICITE FSE available from Sanshin Kohkoh.
*12 Titanium Dioxide (0.040 μm): Titanium Dioxide TTO-55 available from Ishihara.
*13 Ascorbic Acid: Ascorbic Acid available from ROCHE VITAMINS JAPAN K.K.
*14 D-delta-tocopherol: D-DELTA-TOCOPHEROL available from EISAI CO., LTD.
*15 Ethylhexyl Methoxycinnamate: PARSOL MCX available from ROCHE VITAMINS JAPAN K.K.
*16 Butylene Glycol: 1,3-Butylene Glycol available from Celanese.
*17 Titanium Dioxide (0.035 μm): Titanium Dioxide MT500B available from TAYCA.
*18 Mica coated with Titanium Dioxide: FLAMENCO SUPER PEARL available from THE MEARL.
*19 Glucosyl Hesperidin: α -Ghesperidin PS-CC, available from Hayashibara.
*20 Niacinamide: Niacinamide USP available from DSM.
*21 Mulberry Root Extract: Mulberry BG, available from Maruzen Pharmaceuticals.
*22 Panthenol: D-Panthenol USP, available from DSM.
*23 Saccharomycopsis Ferment Filtrate: SK-II Pitera available from Kashiwayama.

### Method of Preparation

The capsules of Examples 1-4 can be made in either of the following 3 methods. Methods 1 and 2 are suitable for small scale production, while Method 3 is suitable for large scale production. In any method, stable fine capsules of loose powder form were obtained, which pass The Drop Test of 100 cm.
1. Components (1)-(10) are mixed and transferred to a container that has a hydrophobic inner surface. Components (16)-(28) are separately mixed and transferred to the same container. The container is closed and shaked by hands for a couple minutes. After naked eye observation of capsules in loose powder form, components (11)-(15) & (29) that had been separately mixed are transferred to the container and mixed.
2. Components (1)-(8) are mixed and transferred to a container that has a hydrophobic inner surface. Components (16)-(28) are separately mixed and transferred to the same container. The container is closed and shaked by hands for a couple minutes. After naked eye observation of capsules in loose powder form, components (9)-(10) that had been separately mixed are transferred to the container and mixed. Then components (11)-(15) & (29) that had been separately mixed are transferred to the container and mixed.
3. Components (1)-(8) are mixed by a pulverizer and transferred to a ribbon blender that has a hydrophobic inner surface. Components (16)-(28) are separately mixed by any conventional method known in the art and sprayed in the ribbon blender. After observation of capsules of water in loose powder form, components (9)-(10) that had been separately mixed are transferred to the ribbon blender and mixed. Then, components (11)-(15) & (29) that had been separately mixed are transferred to the ribbon blender and mixed.

TABLE 2
Composition for Example 5 and Comparative Examples 1-3,
and their Technical Test Results

| | | | Com. | Com. | Com. |
|---|---|---|---|---|---|
| | | Ex. 5 | Ex. 1 | Ex. 2 | Ex. 3 |
| 1 | Titanium Dioxide coated with C9-15 | 10 | | 10 | 10 |
| | fluoroalcohol phosphates (0.25 μm)) *1 | | | | |
| 2 | Nylon-12 coated with C9-15 | 3.5 | 3.5 | | 5 |
| | fluoroalcohol phosphates (5 μm) *9 | | | | |
| 3 | Talc coated with C9-15 fluoroalcohol | | 10 | | |
| | phosphates (9 μm) *24 | | | | |
| 4 | Mica (10 μm) *11 | | | 3.5 | |
| 5 | Sodium Carboxymethyl Starch *7 | 1.5 | 1.5 | 1.5 | |
| 6 | WATER | 85 | 85 | 85 | 85 |
| | Total | 100 | 100 | 100 | 100 |
| | Capsulation | Good | Good | Not | Not |
| | | | | Good | Good |
| | Drop Test | Good | Not | | |
| | | | Good | | |
| | Collapsibility on Application | Good | Good | | |

TABLE 3
Composition for Comparative Examples 4-7, and their Technical
Test Results

| | | Com. | Com. | Com. | Com. |
|---|---|---|---|---|---|
| | | Ex. 4 | Ex. 5 | Ex. 6 | Ex. 7 |
| 1 | Titanium Dioxide coated with C9-15 | 6 | 13 | 5 | 10 |
| | fluoroalcohol phosphates (0.25 μm)) *1 | | | | |
| 2 | Nylon-12 coated with C9-15 | 0.5 | 21 | 5 | 3.5 |
| | fluoroalcohol phosphates (5 μm) *9 | | | | |
| 3 | Talc coated with C9-15 fluoroalcohol | | | | |
| | phosphates (9 μm) *24 | | | | |
| 4 | Porous silica coated with | | | 2.3 | 3 |
| | trimethylsilyl (7 nm, surface area: | | | | |
| | 260 m2/g) *25 | | | | |
| 5 | Sodium Carboxymethyl Starch *7 | 1.5 | 1.5 | 1.5 | 1.5 |
| 6 | WATER | 92 | 64.5 | 86.2 | 82 |
| | Total | 100 | 100 | 100 | 100 |
| | Capsulation | Not | Good | Good | Good |
| | | Good | | | |
| | Drop Test | | Good | Good | Good |
| | Collapsibility on Application | | Not | Not | Not |
| | | | Good | Good | Good |

Definitions of Components
*24 Talc coated with C9-15 fluoroalcohol phosphates (9 μm)): PF-5 TALC JA-46R available from Daito Kasei.
*25 Porous silica coated with trimethylsilyl (7 nm, surface area: 260 m2/g): AEROSIL R812 available from Nippon Aerosil.

### Method of Preparation

Components (1)-(5) were mixed and transferred to a container that has a hydrophobic inner surface. Water (6) was added to the same container. The container was closed and shaked by hands for 2 minutes.

### Methods of Technical Tests

Capsulation: If capsules of even fine particles are observed by the naked eye, evaluation is “Good”. If the capsules are not formed or if the capsules made are not even, evaluation is “Not Good”. Where capsules are not formed, the following tests are not conducted.

Drop Test: Of the capsules obtained, 40 g were tested according to the method described above. If no damage is observed by the naked eye, evaluation is “Good”. If any collapse or agglomeration of the capsule is observed via leaking of liquid or wetting of pigments, evaluation is “Not Good”.

Collapsibility on Application: Collapsibility is evaluated by naked eye observation and sensation upon application on the hand. If the capsule changes to liquid on application accompanying a cooling sensation, evaluation is “Good”. If the capsule does not change to liquid on application, or if there is no cooling sensation, evaluation is “Not Good”.

### Evaluation

The evaluation results of Example 5 and Comparative Example 1-7 are found in Tables 2 and 3. Comparative Example 1, devoid of the first pigment component, was evaluated “No Good” in the Drop Test. Comparative Example 2, devoid of the second pigment component, Comparative Example 3, devoid of gelling agent, and Comparative Example 4, having less than 8% total of the first pigment component and second pigment component, were all evaluated as “Not Good” in Capsulation. Comparative Example 5, having less than 70% water, and Comparative Examples 6 and 7, having 1% or more porous silica having a particle size of less than 1 μm, were all evaluated as “Not Good” in Collapsibility on Application.

### Usage of Examples 1-5

The capsules of Examples 1-5 have appropriate shear tolerance such that it is stable under normal storage conditions as well as normal mixing processes, however, collapses upon a certain shear stress upon application on the skin. When collapsed, the capsules of Examples 1-5 provide good feel to the skin.

Examples 1&2 are useful as foundations. When applied on the skin, the capsules provide good appearance on the skin by balanced coverage and natural look.

Example 3 is useful as a sunscreen. When applied on the skin, the capsules provide good UV protection on the skin with high transparency.

Example 4 is useful as a skin lightening powder. Example 5 is useful as a cooling powder. When applied on the skin, the capsules provide suitable cooling sensation. When applied on the skin, the skin lightening agent of Example 4 is penetrated to the skin.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A collapsible water-containing capsule comprising by weight:
(a) from about 70% to about 92% of a water phase comprising, by weight of the capsule:
(1) from about 5% to about 91.9% water; and
(2) from about 0.1% to about 20% of a gelling agent;
(b) from about 1% to about 29.9% of a first pigment component which has a particle size of less than 1 μm and is surface coated with a lipophobic hydrophobic coating material; and
(c) from about 0.1% to about 29% of a second pigment component which has a particle size of 1 μm or more, is surface coated with a hydrophobic coating material; and is spherical in shape;
wherein the total of the first pigment component and the second pigment component is at least about 8% of the capsule; and
wherein the capsule comprises less than 1% of a porous pigments having a particle size of less than 1 μm.

2. The collapsible water-containing capsule of claim 1 wherein the capsule is substantially free of surfactants.

3. The collapsible water-containing capsule of claim 1 comprising from about 6% to about 27% of a first pigment component.

4. The collapsible water-containing capsule of claim 1 wherein the water phase further comprises a water-soluble solvent.

5. The collapsible water-containing capsule of claim 1 wherein the lipophobic hydrophobic coating material is selected from the group consisting of perfluorooctyl triethoxylsilane, perfluoroalkylphosphoric acids, their salts, and mixtures thereof.

6. The collapsible water-containing capsule of claim 1 wherein the gelling agent is selected from starch derivative polymers.

7. The collapsible water-containing capsule of claim 1 wherein the gelling agent is selected from cellulose derivative polymers.

8. The collapsible water-containing capsule of claim 1 wherein the first pigment component is selected from the group consisting of titanium dioxide, zinc oxide, iron oxide, barium sulfate, polystyrene, and mixtures thereof.

9. The collapsible water-containing capsule of claim 1 wherein the second pigment component is selected from the group consisting of methyl methacrylate copolymer, silica beads, nylon, polymethyl silsesquioxane, vinyl dimethicone/methicone silsesquioxane crosspolymer, polyorganosiloxane elastomer, and mixtures thereof.

10. The collapsible water-containing capsule of claim 1 wherein the collapsible water-containing capsule has appropriate shear tolerance such that it is stable under normal storage conditions as well as normal mixing processes, however, collapses upon application on the personal surface.

11. The collapsible water-containing capsule of claim 8 wherein the collapsible water-containing capsule has a shear tolerance of at least 100 cm according to The Drop Test.

12. The collapsible water-containing capsule of claim 1 further comprising a skin benefit agent.

13. The collapsible water-containing capsule of claim 1 further comprising a perfume.

14. A method of treating or making up of the skin comprising the steps of:
(1) providing the collapsible water-containing capsule of claim 1;
(2) shearing the collapsible water-containing capsule on the skin by a finger or an applicator to allow the collapsible water-containing capsule to collapse; whereby the components of the collapsible water-containing capsule are applied on the skin; and
(3) allowing the water to evaporate and/or be absorbed in the skin.

15. A method of treating or making up of the skin comprising the steps of:
(1) providing the collapsible water-containing capsule of claim 1;
(2) shearing the collapsible water-containing capsule on the skin by a finger or an applicator to allow the collapsible water-containing capsule to collapse; whereby the components are applied on the skin in the order of vicinity from the skin, water, the first pigment component, and the second pigment component; and
(3) allowing the water to evaporate and/or be absorbed in the skin.

16. A process for making the collapsible water-containing capsule of claim 1 comprising the steps of:
(1) uniformly mixing the gelling agent, the first pigment component and the second pigment component; and
(2) mixing the product of step (1) with the water phase.

17. The process for making the collapsible water-containing capsule of claim 1 comprising the steps of:
(1) uniformly mixing the gelling agent and the first pigment component;
(2) mixing the product of step (1) with the water phase; and
(3) mixing the product of step (2) with the second pigment component.
